SEQUENCE LISTING (1 sequence)

SEQ ID NO: 1 - DNA, 32 bp, Artificial
  Synthetic oligonucleotide.
gaattgtaatacgactcactatagggnnnnnn

(19) United States
(12) Patent Application Publication
(10) Pub. No.: US 2005/0106591 A1
(43) Pub. Date: May 19, 2005

(54) Methods and kits for preparing nucleic acid samples

(75) Inventors: John E. Blume, Danville, CA (US); Yanxiang Cao, Mt. View, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/917,643

(22) Filed: Aug. 13, 2004

Related U.S. Application Data
(60) Provisional application No. 60/495,232, filed on Aug. 13, 2003.
(60) Provisional application No. 60/542,933, filed on Feb. 9, 2004.

(51) Int. Cl. C12Q 1/68; C12P 19/34

(52) U.S. Cl. 435/6; 435/91.2

(57) ABSTRACT

The present invention provides methods for preparing nucleic acid samples. The methods of the present invention are particularly amenable for preparing samples that substantially represent the whole transcripts. The method is particularly suitable to use with microarray based expression analysis.

### REFERENCES TO RELATED APPLICATIONS

### PRIORITY CLAIM

The present application claims priority to U.S. Provisional Application Ser. No. 60/495,232, filed on Aug. 13, 2003 and U.S. Provisional Application Ser. No. 60/542,933, filed on Feb. 9, 2004. All cited patent applications are incorporated herein by reference in its entirety for all purposes.

### BACKGROUND OF THE INVENTION

Nucleic acid sample preparation methods have greatly transformed laboratory research that utilize molecular biology and recombinant DNA techniques and have also impacted the fields of diagnostics, forensics, nucleic acid analysis and gene expression monitoring, to name a few. There remains a need in the art for methods that amplify substantially entire transcripts.

### SUMMARY OF THE INVENTION

In one aspect of the invention, methods for preparing nucleic acid samples that represent RNA transcripts are provided. The methods are particularly suitable for preparing samples that are used for detecting transcript features such as exons and alternative splicing. The methods are suitable for quantitative, semi-quantitative or qualitative detection of such transcript features. The methods can be used to monitor a large number transcripts including all types of variants such as alternative spliced transcripts. The methods are particular suitable for microarray based parallel analysis of a large number of, such as more than 1000, 5000, 10,000, 50,000 different target transcripts or transcript features. As used herein, the term “target transcript” or “target nucleic acid” is used to refer to transcripts or other nucleic acids of interest.

In a preferred embodiment, the method for preparing a nucleic acid sample includes hybridizing a primer mixture with a plurality of RNA transcripts or nucleic acids derived from the RNA transcripts and synthesizing first strand cDNAs complementary to the RNA transcripts and second strand cDNAs complementary to the first strand cDNAs, where the primer mixture contains oligonucleotides with a promoter region and a random sequence primer region; and transcribing RNA initiated from the promoter region to produce the nucleic acid sample. The primer region can be a random hexamer. The promoter is typically a prokaryotic promoter such as a bacteriophage promoter, preferably a T7, T3 or SP6 promoter.

The method can be used to analyze eukaryotic mRNA or other RNAs. Total RNA samples or poly(A)+ enriched samples are all suitable for use with this method.

In a particularly preferred method, the resulting cRNA can be used as templates to synthesize second cDNAs. The second cDNA synthesis may be carried out using random primers such as random hexamer.

While the methods of the invention has broad applications and are not limited to any particular detection methods, they are particularly suitable for detecting a large number of, such as more than 1000, 5000, 10,000, 50,000 different transcript features. For example, the second cDNAs may be fragment/labeled and then hybridized with nucleic acids for detection. The labeling steps may be carried out, for example, during cDNA synthesis. Oligonucleotide probes are particularly suitable for detecting specific transcript features such as specific exons and/or splice junctions in transcripts. Typically, a collection of at least 5,000, 10,000, 50,000, 100,000 or 500,000 oligonucleotide probes may be used for detection. The nucleic acid probes may be immobilized on a collection of beads or on a single substrate.

In another aspect of the invention, a reagent kit for the preparing nucleic acid samples is provided. An exemplary reagent kit contains a container comprising an oligonucleotide mixture component and instructions for use of the oligonucleotide mixture where the oligonucleotide in the oligonucleotide mixture component comprises a random primer region and a promoter region. One illustrative oligonucleotide mixture has the sequences of

| | |
|---|---|
| 5′ GAATTGTAATACGACTCACTATAGGGNNNN | (SEQ ID: 01) |
| NN 3′ | |

(NNNNNN represents the random hexamer region)

The reagent kit may further include a container containing a reverse transcriptase and a container containing an RNA polymerase. The kit may have a random primer mixture (such as a random hexamer mixture), in addition to the oligonucleotide mixture with a random primer and a promoter region. Additional components may include labeling and fragmentation reagents, nucleotides, etc.

In a preferred embodiment, the kit include a collection of at least 1000, 5000, 10,000 or 50,000 different nucleic acid probes designed to detect sequences representing target RNA transcripts. The nucleic acid probes may be immobilized on a substrate. They are typically designed to at least 5000 different exons and/or at least 500 splice junctions.

The methods and reagent kits of the invention has extensive applications in biological research, diagnostics, toxicology, drug discovery and other areas.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing a preferred embodiment (small sample WTA or sWTA) employing an oligonucleotide primer that contains a random hexamer (RH) region and -a T7 promoter region. This method has two cDNA synthesis steps. The cDNA can be end labeled at the 5′ or 3′ end or internally labeled.

FIG. 2 is a schematic comparing two protocols, one with one cDNA synthesis step for preparing cRNA samples and the other with two cDNA synthesis steps for preparing cDNA samples. The cRNAs may be fragmented/labeled for hybridization.

FIG. 3 is a schematic showing a random hexamer cDNA protocol for preparing cDNA samples (WTA). Optionally, second strand cDNA may also be synthesized.

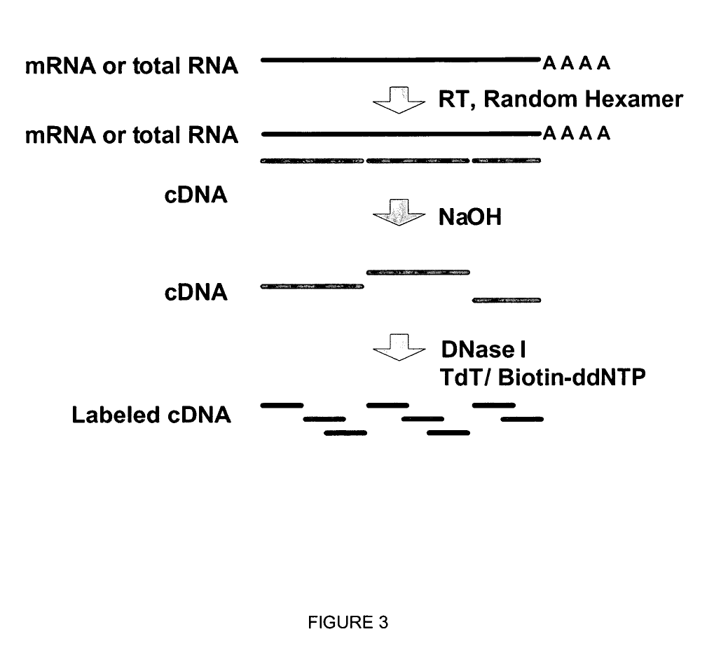

FIG. 4 compares the performance of sWTA and WTA.

FIG. 5 shows that RP-T7-cDNA Amplification (sWTA) protocol is useful for detecting across an exemplary full-length transcript.

### DETAILED DESCRIPTION OF THE INVENTION

In one aspect of the invention, methods and compositions are provided for analyzing RNA transcription. Methods and compositions for preparing nucleic acid samples that are derived from transcript samples are provided. In preferred embodiments, the nucleic acid samples represent the transcript population in the transcript samples. Therefore, these preferred methods are particularly suitable for preparing nucleic acids samples that are used for interrogating transcript feature/structures such as exons structures and splicing in the transcripts. The methods of the invention generally have a better ability to make transcript anywhere across the target, not just at the 3′ or 5′ end. The preferred methods typically include synthesizing nucleic acids using transcripts as templates and random oligonucleotides as primers (e.g., by reverse transcription reactions). The synthesized nucleic acids are then further processed to obtain nucleic acid samples. The methods are particularly useful for microarray based experiments. However, the sample preparation methods may also be used for other detection methods.

In another aspect of the invention, assay kits that contains one or more primers (which may contain a random region and a fixed content region, such as a T7 promoter), optionally contains a reverse transcriptase, RNA polymerase, labeling reagents, and/or fragmentation reagents.

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The present invention may also make use of the several embodiments of the array or arrays and the processing described in U.S. Pat. Nos. 5,545,531 and 5,874,219. These patents are incorporated herein by reference in their entireties for all purposes.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Array Plate or a Plate a body having a plurality of arrays in which each array is separated from the other arrays by a physical barrier resistant to the passage of liquids and forming an area or space, referred to as a well.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs) as described in U.S. Pat. No. 6,156,501 that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Excitation energy refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about IM, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 750 mM NaCl, 50 mM NaPhosphate, 5 mM EDTA, pH 7.4 (5× SSPE) and a temperature of from about 25° C. to about 30° C.

Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning: A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Label for example, a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Microtiter plates are arrays of discrete wells that come in standard formats (96, 384 and 1536 wells) which are used for examination of the physical, chemical or biological characteristics of a quantity of samples in parallel.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 20, 25, 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Reader or plate reader is a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

WGSA (Whole Genome Sampling Assay) Genotyping Technology: A technology that allows the genotyping of thousands of SNPs simultaneously in complex DNA without the use of locus-specific primers. In this technique, genomic DNA, for example, is digested with a restriction enzyme of interest and adaptors are ligated to the digested fragments. A single primer corresponding to the adaptor sequence is used to amplify fragments of a desired size, for example, 500-2000 bp. The processed target is then hybridized to nucleic acid arrays comprising SNP-containing fragments/probes. WGSA is disclosed in, for example, U.S. Provisional Application Ser. Nos. 60/319,685, 60/453,930, 60/454,090 and 60/456,206, 60/470,475, U.S. patent application Ser. Nos. 09/766,212, 10/316,517 ,10/316,629, 10/463,991, 10/321,741, 10/442,021 and 10/264,945, each of which is hereby incorporated by reference in its entirety for all purposes.

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

3. Sample Preparation Methods for Whole Transcript Assays

In one aspect of the invention, methods that are suitable for preparing nucleic acid samples that represent at least 70%, 80%, 90% of the exons of transcripts, or whole transcripts, are provided. In preferred embodiments, the methods are used to prepare nucleic acid samples from at least 70%, 80%, 90% or all exons in a transcript for hybridization with a nucleic acid probe array, such as a high density oligonucleotide array that may contain probes targeting the exons and optionally junctions between exons. The methods of the invention are also particularly suitable for use with tiling arrays such as those described in U.S. patent application Ser. No. 10/815,333, which is incorporated herein. In preferred embodiments, the arrays may have probes that target at least 50%, 70%, 80%, 90% or all the exons of at least 500, 1000, 10,000 transcripts.

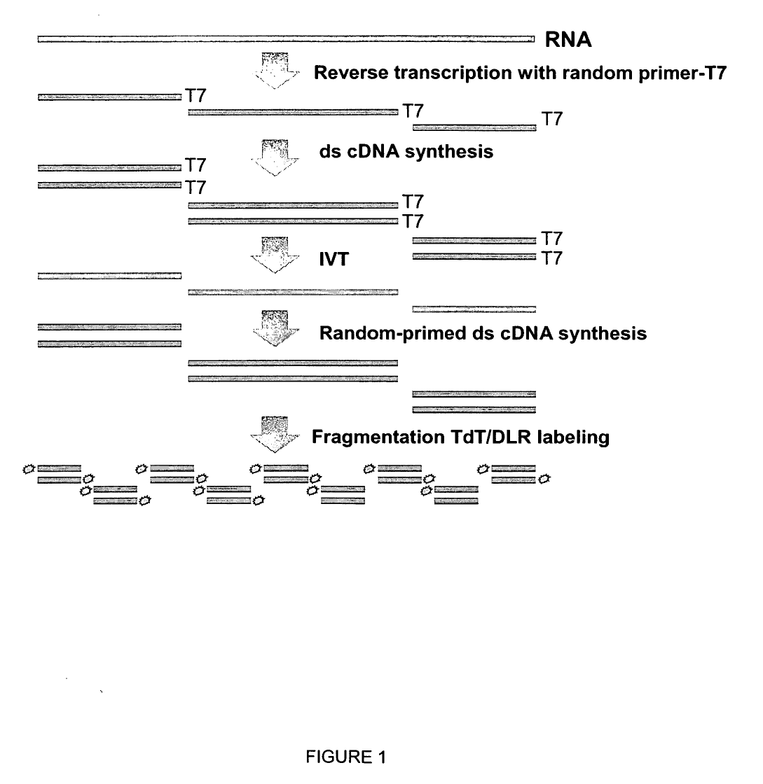

In a preferred embodiment, RNA transcript samples (illustrated in FIG. 1) are used as templates for a reverse transcription reaction to synthesize cDNA. Methods for synthesizing cDNAs are well known in the art. In the preferred embodiments, however, a oligonucleotide primer with a random region and a fixed content region may be used. One exemplary primer is a random hexamer and a T7 promoter that may be useful for later in vitro transcription reactions:

| | |
|---|---|
| 5′ GAATTGTAATACGACTCACTATAGGGNNN | (SEQ ID NO: 01) |
| NNN 3′ | |

(NNNNNN represents the random hexamer region)

The random region is useful for random priming of the primer with the transcript sequences so that the resulting cDNA is more representative of the various regions of the transcripts. In preferred embodiments, the random region of the primer may be 5, 6, 7, 8, 9 bases in length. The fixed content region is typically used to provide a desired function in subsequent reactions. For example, a T7 promoter may be useful for an in vitro transcription reaction. One of skill in the art would appreciate that promoters other than T7, such as T3 and SP6 are also commonly used for in vitro transcription and are suitable for use as the fixed content region. Polymerase for various in vitro transcription promoters are commercially available from, for example, Ambion, Inc. (Austin, Tex., USA).

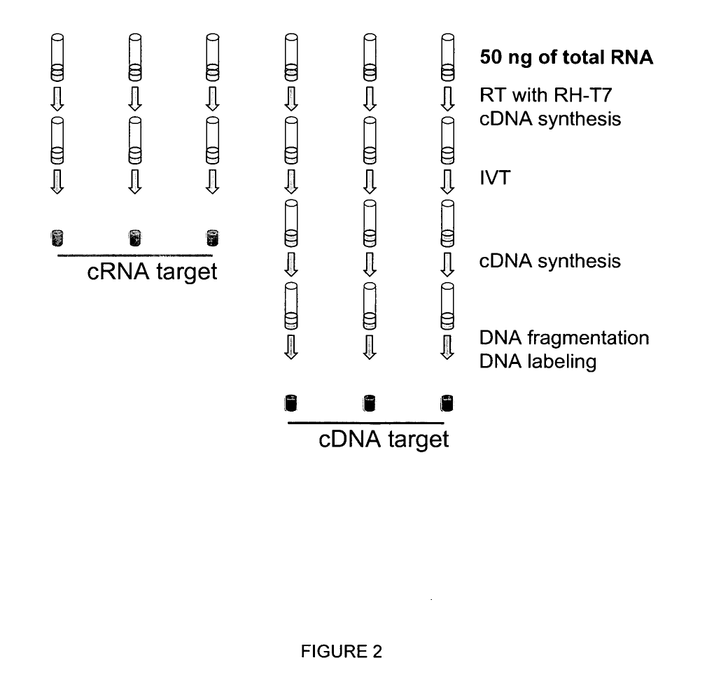

As FIG. 1 shows, the resulting cDNA (typically double stranded) may be used as templates for in vitro transcription reactions to synthesize cRNA. The cRNA targets may be labeled/fragmented for hybridization and detection (see FIG. 2). However, in a particularly preferred embodiment, the cRNAs are used as templates for another cDNA synthesis reaction using, for example, a random primer. The resulting cDNA may be labeled and fragmented for hybridization and detection. This approach typically enhances the detection sensitivity.

FIG. 2 comparing the two approaches. One of skill in the art would appreciate that the invention is not limited to any specific labeling or fragmentation methods. Many suitable labeling and fragmentation methods may be used. Additional DNA fragmentation methods that are suitable for use to enhance hybridization are described in, for example, U.S. Provisional Application Ser. No. 60/589,648, 60/545,417, 60/512,569, 60/506,697, all incorporated herein by reference.

The following is a detailed protocol as a non limiting example to illustrate the preferred embodiment. This exemplary protocol was used to detect transcription features, such as exons, alternative splicing, etc., in several large scale experiments with excellent results (data not shown). Table 1 is a list of exemplary reagents and materials.

TABLE 1

Reagents and Materials

| | | |
|---|---|---|
| REAGENT NAME | VENDOR | P/N |

| | | |
|---|---|---|
| DEPC'ed water, 4 L | Ambion | 9920 |
| DNA-free Total RNA | | |
| Random Primer-T7 (RP-T7), | | |
| 5′ GAATTGTAATACGACTCACTATAG | | |
| GGNNNNNN 3′ | | |
| SuperScript II, 200 U/μL, | Invitrogen | 18064-071 |
| 40,000 U | | |
| 5X First strand buffer and | | |
| 0.1 M DTT included | | |
| dNTP mix, 10 mM, 100 μL | Invitrogen | 18427-013 |
| Superase In, 20 U/μL, | Ambion | 26964 |
| 2,500 U | | |
| Klenow Fragment (3′->5′ | NEB | M0212L |
| exo-), 5 U/μL, 1000 U | | |
| Magnesium Chloride, 25 mM | ABI | |
| (from PCR kit) | | |
| Random Primer, 3 μg/μL, | Invitrogen | 48190-011 |
| 300 μg | | |
| RNase H, 2 U/μL, 120 U | Invitrogen | 18021-071 |
| Large Fragment of DNA | Invitrogen | 18012-039 |
| Polymerase I, 3-9 U/μL, | | |
| 500 U | | |
| DNase I, 1 U/μL, 5,000 μL | Promega | M6101 |
| One-Phor-All plus Buffer, | Amersham | 27-0901-02 |
| 10X | | |
| MEGAscript T7 Kit | Ambion | 1334 |
| RNeasy Mini Kit | Qiagen | 74104 |
| QIAquick PCR Purification | Qiagen | 28104 |
| kit (50) | | |
| Terminal Transferase, | Roche | 3 333 574 |
| recombinant | Diagnostics | |
| 5X Buffer and 25 mM CoCl2 | | |
| included | | |
| DLR-1a, 5 mM | Affymetrix | 900430 |

cRNA Amplification
Step 1. First Strand cDNA Synthesis

1. Mix total RNA sample and RP-T7 primer thoroughly in a 0.2 μL of PCR tube:

| | |
|---|---|
| Total RNA, (10 ng-100 ng) | 1 μL |
| RP-T7 primer, 2 pmol/ng | 1 μL |
| H₂O | 3 μL |
| Total volume | 5 μL |

2. Incubate at 65° C. in thermal cycler for 5 minutes, then keep at 4° C. for 2 minutes, and spin down to collect sample.

3. Prepare the RT_Premix_{—}1 as follows:

| | |
|---|---|
| DEPCed H₂O | 0.5 μL |
| 5 × 1^{st} strand buffer | 2 μL |
| DTT, 0.1 M | 1 μL |
| dNTP mix, 10 mM | 0.5 μL |
| Superase In, 20 U/μL | 0.5 μL |
| SuperScript II, 200 U/μL | 0.5 μL |
| Total volume | 5 μL |

4. Add 5 μL of the RT_Premix_{—}1 to the denatured RNA and primer mixture to make a final volume of 10 μL.

5. Mix thoroughly, spin down, and incubate at 25° C. for 10 minutes, at 37° C. for 1 hour, then keep at 4° C. for no longer than 10 minutes.

Step 2. Second Strand cDNA Synthesis

1. Prepare SS_Premix_{—}1 as follows:

| | |
|---|---|
| DEPC'ed water | 4.575 μL |
| MgCl₂, 25 mM | 2.8 μL |
| Klenow Fragment (exo-), 5 U/μL | 2.5 μL |
| RNase H, 2 U/μL | 0.125 μL |
| Total volume | 10 μL |

2. Add 10 μL of the SS_Premix_{—}1 to each first strand reaction to make a final volume of 20 μL.

3. Mix thoroughly and spin down, then incubate at 37° C. for 50 minutes.

4. Inactive the Klenow Fragment (exo-) at 70° C. for 10 minutes, and keep at 4° C. for no longer then 10 minutes to proceed to the next step.

Step 3. IVT for cRNA Amplification Using Ambion MEGAscript T7 Kit

1. Add the following reagents to the 2nd strand synthesis reaction at room temperature according to the following order:

| | |
|---|---|
| ATP, 75 mM | 5 μL |
| CTP, 75 mM | 5 μL |
| GTP, 75 mM | 5 μL |
| UTP, 75 mM | 5 μL |
| 10 × reaction buffer | 5 μL |
| 10 × Enzyme mix | 5 μL |
| Total volume | 50 μL |

2. Mix thoroughly after adding each reagent and spin briefly. Incubate at 37° C. for 16 hours.

Step 4. cRNA Clean-Up with RNeasy Columns

1. Add 50 μL of RNase-free water to the above cRNA product.

2. Follow the RNeasy Mini Protocol for RNA Cleanup handbook from Qiagen that accompanies the RNeasy Mini Kit for cRNA purification.

3. In the last step of cRNA purification, elute the product with 50μ of RNase-free water.

4. Remove 2 μL of the cRNA and add to 78 μL of water to measure the absorbance at 260 nm to determine the cRNA yield.

5. Use speed vacuum to reduce the volume to 7 μL before proceeding to the next step.

Converting cRNA to Double-Stranded cDNA and Labeling

Step 5. Converting cRNA to First Strand cDNA

1. Mix the cRNA and Random primers thoroughly in a 0.2 □L PCR tube:

| | |
|---|---|
| cRNA, variable | 7 μL |
| Random primers, 3 μg/μL | 1 μL |
| Total volume | 8 μL |

2. Spin briefly and incubating at 70° C. for 5 minutes, at 25° C. for 5 minutes.

3. Prepare RT_Premix_{—}2 as follows:

| | |
|---|---|
| 5 × 1^{st} strand buffer | 4 μL |
| DTT, 0.1 M | 2 μL |
| dNTP mix, 10 mM | 1 μL |
| Superase In, 20 U/μL | 1 μL |
| SuperScript II, 200 U/μL | 4 μL |
| Total volume | 12 μL |

4. Add 12 μL of the RT_Premix_{—}2 to the denatured RNA and primer mixture to make a final volume of 20 μL.

5. Mix thoroughly and spin briefly. Incubate at 25° C. for 5 minutes, then 37° C. for 1 hour, and keep at 4° C. for no longer then 10 minutes.

Step 6. Second Stranded cDNA Synthesis

1. Prepare SS_Premix_{—}2 as follows:

| | |
|---|---|
| DEPC'ed water | 9.9 μL |
| MgCl₂, 25 mM | 5.6 μL |
| Large Fragment, 8.4 U/μL | 4 μL |
| RNase H, 2 U/μL | 0.5 μL |
| Total volume | 20 μL |

2. Add 20 μL of the SS_Premix_{—}2 to each first strand reaction to make a final volume of 40 μL.

5. Mix thoroughly and spin down, then incubate at 37° C. for 40 minutes, and keep at 4° C. for no longer than 10 minutes to proceed to the next step or freeze at −20° C.

Step 7. Double-Stranded cDNA Clean-Up

1. Follow the QIAquick PCR Purification Kit protocol to clean up the double stranded cDNA.

2. In the last step of double stranded cDNA purification, elute the product with 37 μL of EB Buffer.

3. Remove 2 μL of the cDNA elute and add to 78 μL of water to measure the absorbance at 260 nm to determine the cDNA yield.

Step 8. Double Stranded cDNA Fragmentation

1. Dilute the 1 U/μL of DNAse I to 0.2 U/□L using 1× One-Phor-All buffer plus.

2. Prepare the following mix:

| | |
|---|---|
| 10 × One-Phor-All buffer plus | 3.6 μL |
| ds cDNA | 30 μL |
| DNAse I (0.2 U/□L) | 3 μL |
| Total volume | 36.6 μL |

3. Spin briefly and incubating at 37° C. for 10 minutes and inactivate the DNase I at 95° C. for 10 minutes, then keep at 4° C.

4. Take 1 μL of the fragmented cDNA to check the size with RNA nano kit on Agilent 2100 Bioanalyzer following the kit instruction. The desirable fragment size should be in 50 to 200 bp range. If necessary, use additional DNase I to obtain the desirable size.

Step 9. Fragmented cDNA Labeling:

1. Prepare the Labeling mix as follows:

| | |
|---|---|
| 5 × TdT Reaction buffer | 14 μL |
| CoCl₂, 25 mM | 14 μL |
| DLR-1a, 5 mM | 1 μL |
| Terminal Transferase, rec (400 U/□L) | 4.4 μL |
| Total Volume | 33.4 μL |

2. Add 33.4 μL of the labeling mix to 35.6 μl of the fragmented cDNA to make a final volume of 69 μL.

3. Mix and spin briefly. Incubate at 37° C. for 60 minutes, and keep at 4° C.

Step 10. Hybridization

1. Prepare the Hybridization Mix as follows:

| | |
|---|---|
| 2 × MES Hybridization buffer | 100 μL |
| Control Oligo B2, 3 mM | 3 μL |
| 20 × RNA control | 10 μL |
| BSA, acetelated, 50 mg/μL | 2 μL |
| Herring sperm DNA, 10 mg/μL | 2 μL |
| DMSO, 100% | 14 μL |
| Total volume | 131 μL |

2. Add 131 μL of the Hybridization Mix to 69 μL of the labeling reaction to make a final volume of 200 μL, mix well and denature at 99° C. for 10 minutes and keep at 50° C. for 5 minutes in a thermal cycler.

3. Hybridize the 200 μL of the labeled cDNA to pre-wetted GeneChip® probe array (cDNA test array) at 50° C. for 16 hours.

4. Follow the wash and scan procedures described in the GeneChip® Expression Analysis Technical Manual (Affymetrix, Santa Clara, Calif., USA), incorporated herein by reference.

FIG. 2 shows another protocol for whole transcript analysis. This WTA protocol is based upon random primer cDNA synthesis. A detailed protocol is provided herein as a non limiting example:

cDNA Target Preparation

Reagents and Materials

Random Primers, 3 μg/μL, Invitrogen Life Technologies, P/N 48190-011
SuperScript II Reverse Transcriptase, Invitrogen Life Technologies, P/N 18064-071
SUPERase•In™, Ambion, P/N 2696
NaOH, 1 N solution, VWR Scientific Products, P/N MK469360
HCl, 1 N solution, VWR Scientific Products, P/N MK638860
QIAquick PCR Purification Kit, QIAGEN, P/N 28104
10× One-Phor-All Buffer, Amersham Pharmacia Biotech, P/N 27-0901-02
Deoxyribonuclease I (DNase I), Amersham Pharmacia Biotech, P/N 27-0514-01
EDTA, 0.5 M pH 8.0, Invitrogen Life Technologies, P/N 15575-020
Terminal Transferase (including buffer and CoCl₂), 400 U/μL, recombinant, Roche Applied Science,
   P/N 3 333 574
DLR-1a, 5 mM, Affymetrix, P/N 900430
   cDNA Synthesis

The starting material for the following protocol is 5 μg of total RNA. Incubations are performed in a thermocycler.

Step 1: cDNA Synthesis

1. Prepare the following mixture for primer annealing:
Dilute Random Primer from 3 μg/μL to 750 ng/μL (1:4 dilution).

RNA/Primer Annealing Mix

| | | | Final |
|---|---|---|---|
| Components | Volume | | Concentration |
| Total RNA | 5 | μg | — |
| Random Primer (750 ng/ul) | 1 | μL | 25 ng/μL |
| Nuclease-free H₂O | up to 30 | μL | — |
| Total Volume Added | 30 | μL | |

2. Incubate the RNA/Primer mix at the following temperatures:
   70° C. for 10 minutes
   25° C. for 10 minutes
   Chill to 4° C.
3. Prepare the reaction mix for cDNA synthesis. Briefly centrifuge the reaction tube to collect sample at the bottom and add the cDNA synthesis mix from following table to the RNA/primer annealing mix.

cDNA Synthesis Components

| | | Final |
|---|---|---|
| Components | Volume | Concentration |
| RNA/Primer Annealing Mix | 30 μL | |
| 5 × 1st Strand Buffer | 12 μL | 1× |
| 100 mM DTT | 6 μL | 10 mM |
| 10 mM dNTP | 3 μL | 0.5 mM |
| SUPERase.In (20 U/ul) | 1.5 μL | 0.5 U/μL |
| SuperScript II (200 U/ul) | 7.5 μL | 25 U/μL |
| Total Volume | 60 μL | |

4. Incubate the reaction at the following temperatures:
   25° C. for 10 minutes
   37° C. for 60 minutes
   42° C. for 60 minutes
   Inactivate SuperScript II at 70° C. for 10 minutes
   Chill to 4° C.
      Step 2: Removal of RNA
1. Add 20 μL of 1 N NaOH and incubate at 65° C. for 30 minutes.
2. Add 20 μL of 1 N HCl to neutralize.
   Step 3: Purification and Quantitation of cDNA Synthesis Products
1. Use QIAquick Column to clean up the cDNA synthesis product (for detailed protocol, see QIAquick PCR Purification Kit Protocols provided by the supplier). Elute the product with 40 μL of EB Buffer (supplied with QIAquick kit).
2. Take 2 ul from above elution and quantify the purified cDNA product by 260 nm absorbance (1.0 A₂₆₀ unit=33 μg/mL of single strand DNA).
   cDNA Fragmentation
   1. Prepare the following reaction mix:

Fragmentation Reaction Mix

| | | | Final |
|---|---|---|---|
| Components | Volume | | Concentration |
| 10 × One Phor-All Buffer | 4.5 | μL | 1× |
| cDNA template | all (˜38 | μL) | 1.5˜5 μg |
| Dnase I (see note below) | X | μL | 0.6 U/μg of cDNA |
| Nuclease-free H₂O | up to 45 | μL | |
| Total Volume | 45 | μL | |

2. Incubate the reaction at 37° C. for 10 minutes.
3. Inactivate DNase I at 98° C. for 10 minutes.
4. The fragmented cDNA is applied directly to the terminal labeling reaction. Alternatively, the material can be stored at −20° C. for later use.
   Terminal Labeling

Use Roche Terminal Transferase, recombinant with DLR-1a (Affymetrix, Santa Clara, Calif., USA) to label the 3′ termini of the fragment products.
1. Prepare the following reaction mix:

Terminal Label Reaction

| | | | Final |
|---|---|---|---|
| Components | Volume | | Concentration |
| 5 × TdT Reaction Buffer | 14 | μL | 1× |
| 25 mM CoCl2 | 14 | μL | 5 mM |
| rTDT (400 U/ul) | 4.375 | μL | 5.8 U/pmol |
| cDNA template (1.5-5 ug) | 37 | μL | |
| DLR-1a (5 mM) | 1 | μL | 0.07 mM |
| Total Volume | ˜70 | μL | |

2. Incubate the reaction at 37° C. for 60 minutes.
3. Stop the reaction by adding 2 μL of 0.5 M EDTA (PH 8.0).
4. The target is ready to be hybridized onto probe arrays. Alternatively, it may be stored at −20° C. for later use.
   Target Hybridization
   Reagents and Materials
   2× MES Hybridization Buffer (See GeneChip® Expression Analysis Technical Manual for preparation)
   Acetylated Bovine Serum Albumin (BSA) solution, 50 mg/mL, Invitrogen Life Technologies, P/N 15561-020
   Herring Sperm DNA, 10 mg/mL, Promega Corporation, P/N D1811
   GeneChip Eukaryotic Hybridization Control Kit, Affymetrix, P/N900299
   Control Oligo B2, 3 nM, Affymetrix, P/N 900301 (can be ordered separately)
   100% DMSO, Sigma, P/N D-4818
      Target Hybridization
1. Mix the following for each target, scaling up volumes for hybridization to multiple probe arrays.

Hybridization Cocktail for Single Midi Probe Array

| | | | Final |
|---|---|---|---|
| Components | Volume | | Concentration |
| 2 × MES Hybridization | 100 | μl | 1× |
| Buffer | | | |
| Control Oligo B2 | 3.3 | μL | 50 pM |
| 20 × Spike Controls | 10 | μL | 1× |
| HS DNA (10 mg/ml) | 2 | μL | 0.1 mg/ml |
| Ace-BSA (50 mg/ml) | 2 | μL | 0.5 mg/ml |
| 100% DMSO | 14 | μL | 7% |
| Fragmented cDNA | 70 | μL | — |
| Total Volume | ˜200 | μL | |

2. Equilibrate probe array to room temperature immediately before use.
3. Heat the hybridization cocktail to 99° C. for 5 minutes and hold it at 50° C.
4. Meanwhile, wet the array by filling it with 1× Hybridization Buffer. Incubate the probe array at 50° C. for 10 minutes with rotation.
5. Spin hybridization cocktail at maximum speed to remove any insoluble material.
6. Remove the buffer solution from the probe array and fill with hybridization cocktail.
7. Place probe array in the rotisserie box in 50° C. oven, rotate at 60 rpm, and hybridize for 16 hours.
   Probe Array Wash and Stain
   Reagents and Materials
   2× MES Stain Buffer (See GeneChip Expression Analysis Technical Manual for preparation)
   Acetylated Bovine Serum Albumin (BSA) solution, 50 mg/mL, Invitrogen Life Technologies, P/N 15561-020
   R-Phycoerythrin Streptavidin, Molecular Probes, P/N S-866
   Goat IgG, Reagent Grade, Sigma-Aldrich, P/N I 5256
   Anti-streptavidin antibody (goat), biotinylated, Vector Laboratories, P/N BA-0500
      Preparation of Staining Reagents

SAPE Solution Mix for First and Third Stain

| | | Final |
|---|---|---|
| Components | Volume | Concentration |
| 2 × MES Stain Buffer | 600.0 μL | 1× |
| 50 mg/ml BSA | 48.0 μL | 2 mg/ml |
| 1 mg/ml Streptavidin | 12.0 μL | 10 μg/ml |
| Phycoerythrin | | |
| DI H₂O | 540.0 μL | — |
| Total Volume | 1200.0 μL | |

Antibody Solution Mix for Second Stain

| | | Final |
|---|---|---|
| Components | Volume | Concentration |
| 2 × MES Stain Buffer | 300.0 μL | 1× |
| 50 mg/ml BSA | 24.0 μL | 2 mg/ml |
| 10 mg/ml Normal Goat IgG | 6.0 μL | 0.1 mg/ml |
| 0.5 mg/ml Biotin Anti- | 6.0 μL | 5 μg/ml |
| streptavidin | | |
| DI H₂O | 264.0 μL | — |
| Total Volume | 600.0 μL | |

Wash and Stain the Probe Array

Follow the instructions described in the GeneChip® Expression Analysis Technical Manual for the washing and staining steps for eukaryotic targets.

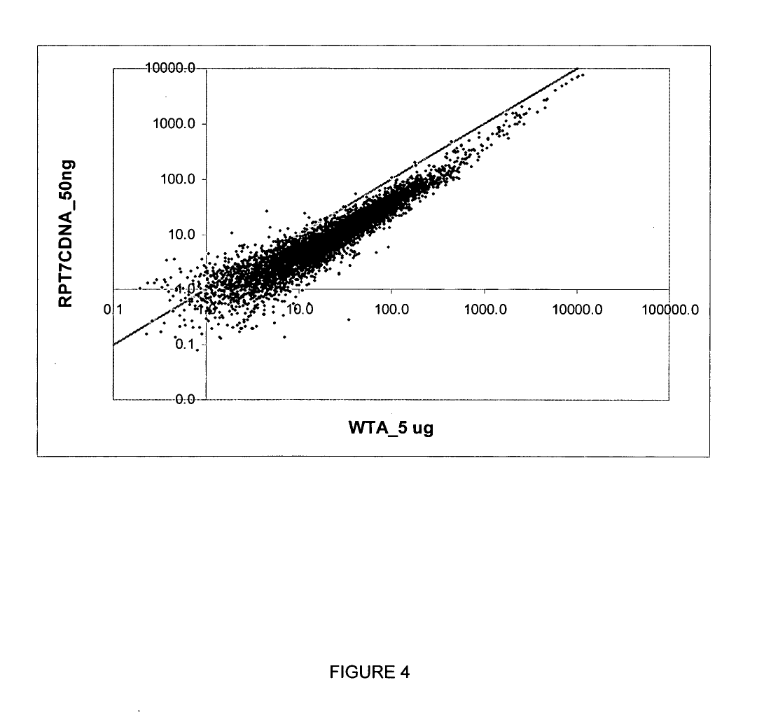

FIG. 4 shows a comparison of probe intensities between random hexamer cDNA protocol (WTA) and sWTA (random/T7 primer, cDNA sample). The sWTA protocol has a good correlation with the WTA protocol (R=0.961±0.004). The detection call concordance was around 90% in the experiment wherein the two protocols are used to detect transcription.

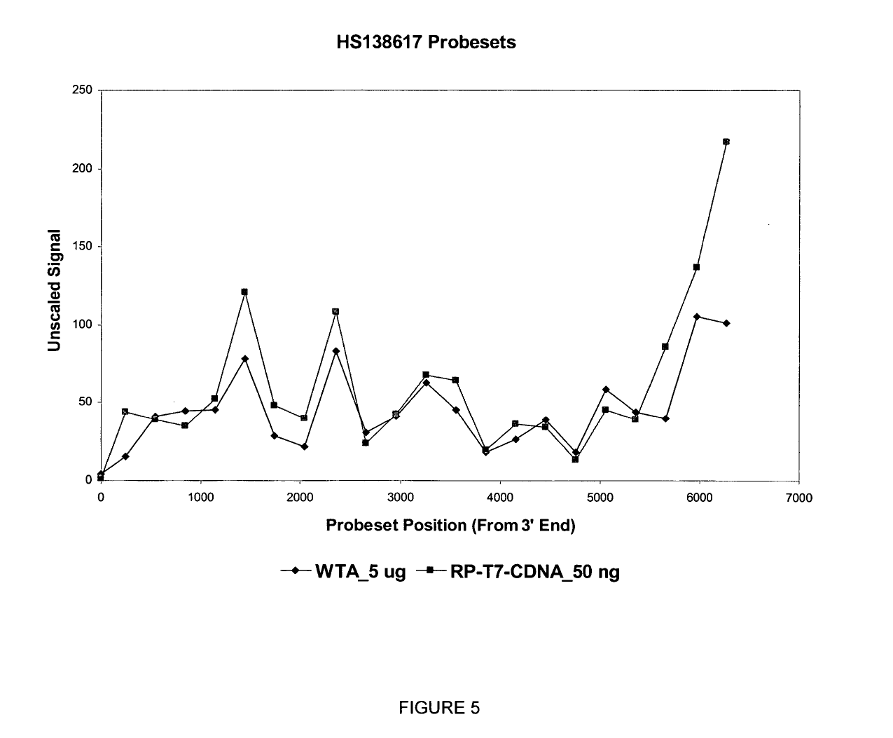

FIG. 5 shows the comparison of WTA protocol and sWTA protocol for detecting an exemplar transcript with probes that are designed to interrogate across the length of the transcript. It can be seen that the two protocols can produce nucleic acid samples that are representing the entire length of the transcript.

In one aspect of the invention, methods for preparing nucleic acid samples that represent RNA transcripts are provided. The methods are particularly suitable for preparing samples that are used for detecting transcript features such as exons and alternative splicing. The methods are suitable for quantitative, semi-quantitative or qualitative detection of such transcript features. The methods can be used to monitor a large number transcripts including all types of variants such as alternative spliced transcripts. The methods are particular suitable for microarray based parallel analysis of a large number of, such as more than 1000, 5000, 10,000, 50,000 different target transcripts or transcript features. As used herein, the term “target transcript” or “target nucleic acid” is used to refer to transcripts or other nucleic acids of interest.

In a preferred embodiment, the method for preparing a nucleic acid sample includes hybridizing a primer mixture with a plurality of RNA transcripts or nucleic acids derived from the RNA transcripts and synthesizing first strand cDNAs complementary to the RNA transcripts and second strand cDNAs complementary to the first strand cDNAs, where the primer mixture contains oligonucleotides with a promoter region and a random sequence primer region; and transcribing RNA initiated from the promoter region to produce the nucleic acid sample. The primer region can be a random hexamer. The promoter is typically a prokaryotic promoter such as a bacteriophage promoter, preferably a T7, T3 or SP6 promoter.

The method can be used to analyze eukaryotic mRNA or other RNAs. Total RNA samples or poly(A)+ enriched samples are all suitable for use with this method.

In a particularly preferred method, the resulting cRNA can be used as templates to synthesize second cDNAs. The second cDNA synthesis may be carried out using random primers such as random hexamer.

While the methods of the invention has broad applications and are not limited to any particular detection methods, they are particularly suitable for detecting a large number of, such as more than 1000, 5000, 10,000, 50,000 different transcript features. For example, the second cDNAs may be fragment/labeled and then hybridized with nucleic acids for detection. Oligonucleotide probes are particularly suitable for detecting specific transcript features such as specific exons and/or splice junctions in transcripts. Typically, a collection of at least 5,000, 10,000, 50,000, 100,000 or 500,000 oligonucleotide probes may be used for detection. The nucleic acid probes may be immobilized on a collection of beads or on a single substrate.

In another aspect of the invention, a reagent kit for the preparing nucleic acid samples is provided. An exemplary reagent kit contains a container comprising an oligonucleotide mixture component and instructions for use of the oligonucleotide mixture where the oligonucleotide in the oligonucleotide mixture component comprises a random primer region and a promoter region. One illustrative oligonucleotide mixture has the sequences of

| | |
|---|---|
| 5′ GAATTGTAATACGACTCACTATAGGGNNN | (SEQ ID NO.: 01) |
| NNN 3′ | |

(NNNNNN represents the random hexamer region)

The reagent kit may further include a container containing a reverse transcriptase and a container containing an RNA polymerase. The kit may have a random primer mixture (such as a random hexamer mixture), in addition to the oligonucleotide mixture with a random primer and a promoter region. Additional components may include labeling and fragmentation reagents, nucleotides, etc.

In a preferred embodiment, the kit include a collection of at least 1000, 5000, 10,000 or 50,000 different nucleic acid probes designed to detect sequences representing target RNA transcripts. The nucleic acid probes may be immobilized on a substrate. They are typically designed to at least 5000 different exons and/or at least 500 splice junctions.

The methods and reagent kits of the invention has extensive applications in biological research, diagnostics, toxicology, drug discovery and other areas. In an exemplary embodiment, transcription of individual exons and splice junction structures are monitored in samples treated with drug candidates. The response of transcription features, such as alternative splicing, to the drug treatment may be analyzed to evaluate the drug candidates. The methods and kits of the invention are particularly suitable for such application because the resulting nucleic acids are more representative of the entire transcript rather than being limited to the 3′ or 5′ region of the transcripts.

In another exemplary application, the methods and kits may be used to process tissue samples to obtain nucleic acid samples. The samples are analyzed for alternatively spliced transcripts. It is well known that alternative splicing is often involved in the pathogenesis of certain diseases. By analyzing the alternative splicing events in the tissue sample, diagnostic information can be obtained.

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

1. A method for preparing a nucleic acid sample comprising:
hybridizing a primer mixture with a plurality of RNA transcripts or nucleic acids derived from the RNA transcripts and synthesizing first strand cDNAs complementary to the RNA transcripts and second strand cDNAs complementary to the first strand cDNAs, wherein the primer mixture comprises oligonucleotides with a promoter region and a random sequence primer region; and
transcribing RNA initiated from the promoter region to produce the nucleic acid sample.

2. The method of claim 1 wherein the random sequence primer region is a random hexamer.

3. The method of claim 2 wherein the promoter region comprises a prokaryotic promoter.

4. Thethod of claim 2 wherein the promoter region comprises a bacteriophage promoter.

5. The method of claim 4 wherein the promoter region comprises a T7 promoter.

6. The method of claim 4 wherein the promoter region comprises a T3 promoter.

7. The method of claim 4 wherein the promoter region comprises a SP6 promoter.

8. The method of claim 1 wherein the RNA transcripts are eukaryotic mRNA.

9. A method for preparing a nucleic acid sample comprising:
hybridizing a primer mixture with a plurality of RNA transcripts or nucleic acids derived from the RNA transcripts and synthesizing first strand cDNAs complementary to the RNA transcripts and second strand cDNAs complementary to the first strand cDNAs to produce first cDNAs, wherein the primer mixture comprises oligonucleotides with a promoter region and a random sequence primer region;
transcribing RNA initiated from the promoter region to produce cRNAs;
hybridizing a random primer mixture with the cRNAs; and
synthesizing second cDNAs from the random primers.

10. The method of claim 9 wherein the random sequence primer region is a random hexamer.

11. The method of claim 10 wherein the promoter region comprises a prokaryotic promoter.

12. The method of claim 10 wherein the promoter region comprises a bacteriophage promoter.

13. The method of claim 12 wherein the promoter region comprises a T7 promoter.

14. The method of claim 12 wherein the promoter region comprises a T3 promoter.

15. The method of claim 12 wherein the promoter region comprises a SP6 promoter.

16. The method of claim 9 wherein the RNA transcripts are eukaryotic mRNA.

17. The method of claim 9 wherein the random primer is a random hexamer.

18. A method for analyzing a plurality of transcripts comprising:
hybridizing a primer mixture with the plurality of RNA transcripts or nucleic acids derived from the RNA transcripts and synthesizing first strand cDNAs complementary to the RNA transcripts and second strand cDNAs complementary to the first strand cDNAs to produce first cDNAs, wherein the primer mixture comprises oligonucleotides with a promoter region and a random sequence primer region;
transcribing RNA initiated from the promoter region to produce cRNAs;
hybridizing a random primer mixture with the cRNAs;
synthesizing second cDNAs from the random primers;
fragmenting the second cDNAs to produce fragmented cDNAs;
hybridizing fragmented cDNAs with a plurality of nucleic acid probes to detect the nucleic acids representing target transcripts.

19. The method of claim 18 wherein the random sequence primer region is a random hexamer.

20. The method of claim 18 wherein the promoter region comprises a prokaryotic promoter.

21. The method of claim 21 wherein the promoter region comprises a bacteriophage promoter.

22. The method of claim 21 wherein the promoter region comprises a T7 promoter.

23. The method of claim 21 wherein the promoter region comprises a T3 promoter.

24. The method of claim 21 wherein the promoter region comprises a SP6 promoter.

25. The method of claim 18 wherein the RNA transcripts are eukaryotic mRNA.

26. The method of claim 18 wherein the random primer is a random hexamer.

27. The method of claim 18 wherein the plurality of nucleic acid probes are immobilized.

28. The method of claim 27 wherein the plurality of nucleic acid probes comprises at least 50,000 probes.

29. The method of claim 28 wherein the plurality of nucleic acid probes are immobilized on a collection of beads, wherein each bead contains a unique probe or probe mixture.

30. The method of claim 28 wherein the plurality of nucleic acid probes are immobilized on a substrate.

31. The method of claim 28 wherein the nucleic acid probes are oligonucleotide probes.

32. The method of claim 31 further comprising labeling the second cDNA fragments before hybridization.

33. The method of claim 32 further comprising labeling the second cDNAs before fragmenting.

34. The method of claim 32 wherein the target transcripts comprise at least 5000 different exons.

35. The method of claim 32 wherein the target transcripts comprises at least 500 splice junctions

36. A reagent kit for the preparing nucleic acid samples comprising: a container comprising an oligonucleotide mixture component and instructions for use of the oligonucleotide mixture wherein the oligonucleotide in the oligonucleotide mixture component comprises a random primer region and a promoter region.

37. The reagent kit of claim 36 further comprising a container comprising a reverse transcriptase and a container comprising an RNA polymerase.

38. The reagent kit of claim 37 further comprising a random primer mixture.

39. The reagent kit of claim 38 wherein the random primer mixture is a random hexamer mixture.

40. The reagent kit of claim 39 further comprising a labeling reagent.

41. The reagent kit of claim 40 further comprising a collection of nucleic acid probes designed to detect sequences representing target RNA transcripts.

42. The reagent kit of claim 42 wherein the collection of nucleic acid probes are immobilized on a substrate.

43. The reagent kit of claim 42 wherein the nucleic acid probes are oligonucleotide probes.

44. The reagent kit of claim 43 wherein the oligonucleotide probes targets at least 5000 different exons.

45. The reagent of claim 43 wherein the oligonucleotide probes target at least 500 splice junctions.
